(19) United States
(12) Patent Application Publication
(10) Pub. No.: US 2020/0368751 A1
(43) Pub. Date: Nov. 26, 2020

(54) THERMAL CYCLER

(71) Applicant: BiOptic (Jiangsu) Inc., Jiangsu (CN)

(72) Inventor: Shou-Kuan Tsai, Jiangsu (CN)

(21) Appl. No.: 16/966,335

(22) PCT Filed: Mar. 27, 2019
(86) PCT No.: PCT/CN2019/079892

§ 371 Date: Jul. 30, 2020

(30) Foreign Application Priority Data
Feb. 13, 2018 (CN) 201810148629.9

(51) Int. Cl. B01L 7/00; C12Q 1/686

(52) U.S. Cl. CPC B01L 7/52; C12Q 1/686; B01L 2200/14; B01L 2300/025; B01L 2300/0627; B01L 2200/145; B01L 2300/02; B01L 2300/06

(57) ABSTRACT

Used for running a polymerase chain reaction, a thermal cycler includes a main body having a chamber, and inside of the chamber is provided with a protocol running device port and a sample block for placing reaction tubes. When a protocol running device is put into the protocol running device port, the main body may execute a thermal cycle onto the reaction tubes according to a protocol stored in the protocol running device.

### CROSS-REFERENCE TO RELATED APPLICATIONS

This application is an application under 35 U.S.C. 371 of International Application No. PCT/CN2019/079892 filed on Mar. 27, 2019, which claims priority of Chinese Patent Application No. 201810148629.9 filed Feb. 13, 2018. Each of the above-referenced applications is expressly incorporated herein by reference, in their entireties.

### BACKGROUND OF THE INVENTION

### 1. Field of the Invention

The present invention relates to a thermal cycler, and more particularly to a thermal cycler for carrying out a polymerase chain reaction.

### 2. Description of Related Art

A conventional polymerase chain reaction (PCR) instrument uses a polymerase chain reaction to replicate a large amount of DNA segments in vitro. In the process, researchers usually inject a solution containing DNA samples and primers into reaction tubes. Amplified DNA fragments are obtained after a few heating and cooling cycles applied to the tubes to perform steps of separation of two strands of the DNA double helix, bind of primers and single-stranded DNA templates, and DNA replication etc. Therefore, the setting of discrete high, low, and medium temperatures and the durations in each step are very important for the polymerase chain reaction instrument to obtain the proper yield of correct DNA.

In current polymerase chain reaction instruments, researchers must employ a user interface provided by the polymerase chain reaction instrument or a remote computer interface connected the PCR instrument to set the temperatures and durations in each step of the thermal cycle before running continuous reactions. The polymerase chain reaction is carried out after the real-time input of temperatures and durations. However, one of the risks of the real-time input is that negligence may cause the polymerase chain reaction to be carried out at the wrong temperature and duration. And this failure caused by wrong temperature and duration may not be discovered until the subsequent inspection of the product, resulting in the wasting of time and samples. Secondly, the real-time input needs to be operated by researchers or persons with professional knowledge in this field, limiting the applicability of the polymerase chain reaction instrument. In addition, the polymerase chain reaction instrument with the connected computer interface limits the environment of use. Therefore, a need is arisen to improve the above deficiencies.

### SUMMARY OF THE INVENTION

The present invention provides a thermal cycler having a device port capable of placing a protocol running device. By placing a protocol running device pre-written with a protocol into the thermal cycler, it is convenient for the user to operate the PCR instrument and to reduce the input error while setting the thermal cycler.

The present invention provides a thermal cycler that employs a protocol running device with a pre-written protocol to set reaction conditions of the thermal cycle. Therefore, the thermal cycler can operate stand-alone without connecting to a network and a remote computer system. It also takes into account user-friendly operation and tracking of reaction conditions.

The present invention provides a thermal cycler, which provides a closed heating environment in a sample block. A heating member above the reaction tubes in the sample block is used to pressurize the reaction tubes, such that condensation in the reaction tubes can be avoided and the concentration of the reaction mixtures in the reaction tubes can be increased.

According to the above, a thermal cycler is provided with: a main body including a chamber and a sample block in the chamber for placing a plurality of reaction tubes; and, a protocol running device port disposed in the chamber to place a protocol running device, wherein the main body executes a thermal cycle on the reaction tubes according to a protocol stored in the protocol running device placed in the protocol running device port.

In one embodiment, the thermal cycler further includes a lid that presses against the main body to seal the chamber between the lid and the main body.

In one embodiment, the lid includes a heating member, and when the lid presses against the main body and reaction tubes are placed in the sample block, the heating member is located above the sample block and the reaction tubes to heat the sample block and to pressurize the reaction tubes.

In one embodiment, the lid includes a locking sensor to detect whether the lid is closed or is locked with the main body.

In one embodiment, the protocol includes a setting of a plurality of temperatures and durations required to carry out a polymerase chain reaction, or the protocol further includes a setting to interrupt or stop the thermal cycle in response to a detecting result of the locking sensor.

In one embodiment, the protocol running device includes an erasable and programmable read-only memory, a flash memory, or a radio frequency identification tag to store the protocol.

In one embodiment, the main body further includes a main screen disposed on the main body to display the protocol.

In one embodiment, the main body further includes a start key disposed on the main body, and the start key is used to start, interrupt, or stop the thermal cycle.

### BRIEF DESCRIPTION OF THE DRAWINGS

FIG. 1 is a front perspective schematic view of a thermal cycler in accordance with an embodiment of the present invention.

FIG. 2 is another front perspective schematic view of a thermal cycler in accordance with an embodiment of the present invention.

### DETAILED DESCRIPTION OF THE PREFERRED EMBODIMENT

The term “thermal cycler” as used herein refers to a instrument configured to perform a reaction composed of a thermal cycle, which consists of a plurality of steps of temperature rise and fall. The durations of the temperature rise and fall may be the same or different, and usually high temperature and low temperature alternate. Accordingly, the present invention uses polymerase chain reaction as an example, but it is not intended to limit the application of the present invention.

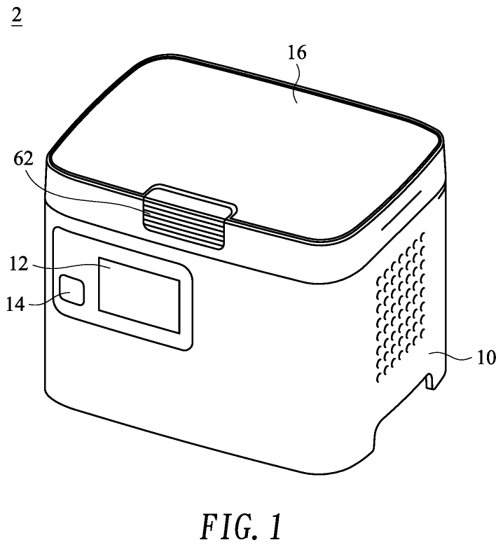

FIG. 1 is a front perspective schematic view of a thermal cycler in accordance with an embodiment of the present invention. The thermal cycler 2 mainly includes two parts, a main body 10 and a lid 16 to cover the main body 10. The main body 10 is provided with a main screen 12 for displaying information such as parameters or results. The main screen 12 can display a temperature before the protocol is loaded and executed, and can display a protocol number/ID, a heating temperature, and an elapsed time and/or a remaining time of the protocol execution during the loading and execution of the protocol. However, the displayed information is not limited to the above.

The main body 10 is also provided with a start key 14 allowing the user for loading, executing, interrupting, or stopping the protocol, or switching the items etc. In addition, a lid 16 covers the main body 10 of the thermal cycler 2 to isolate the outside from the chamber of the main body 10. In the present invention, the lid 16 includes a heating member to prevent condensation in the reaction tubes during the thermal cycle. The lid 16 is provided with a button 62 so that the user can press the button 62 to open the lid 16 and expose the chamber of the main body 10.

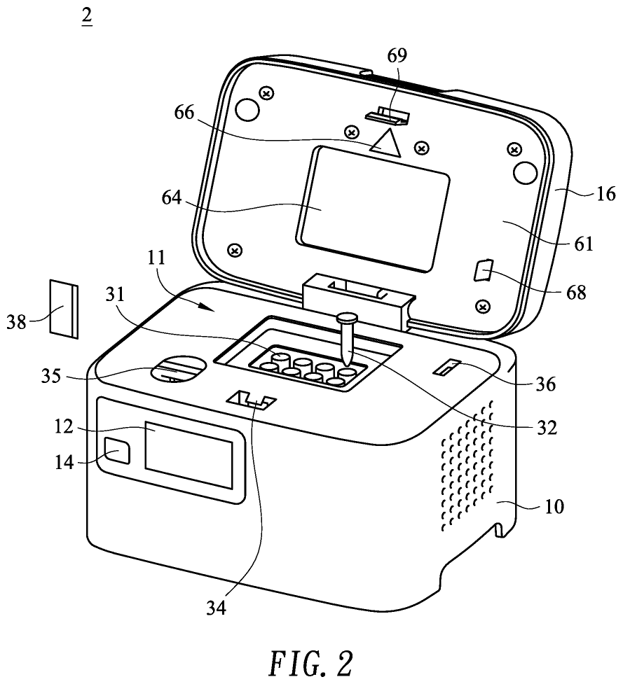

FIG. 2 is another front perspective schematic view of a thermal cycler in accordance with an embodiment of the present invention. Referring to FIGS. 1 and 2, the user presses the button 62 of the lid 16 to open the lid 16 and expose the chamber 11 of the main body 10. In addition, a heating member 64 is provided at the lid 16 and used to heat the reaction tubes 32 placed in the chamber 11. In one embodiment, the heating member 64, such as a heater platen, is at least partially exposed on the first surface 61 of the lid 16 facing the chamber 11, and the exposed heating member 64 may be close to the reaction tubes 32 to directly transfer heat to the reaction tubes 32 placed in the chamber 11. Optionally, a temperature warning 66 may be provided on the first surface 61 of the lid 16 to remind the user to pay attention to the high-temperature chamber 11 after the lid 16 is opened. Moreover, a first latch member 69 and a protruding member 68 are provided on the first surface 61 of the lid 16. The first latch member 69 can fit with a second latch member 34 of the chamber 11 to lock the closed lid 16 and the main body 10. In addition, when the lid 16 and the main body 10 are locked, the protrusion member 68 intervene in a locking sensor 36 of the chamber 11 to confirm the locking of the lid 16 and the main body 10.

With continued reference to FIG. 2, a sample block 31 is disposed in the chamber 11 and is exposed corresponding to the exposed heating member 64 at the first surface 61 of the lid 16. A plurality of reaction tubes 32 can be placed in the sample block 31 and heated. When the lid 16 is pressed against the main body 10, the heating member 64 and the sample block 31 cooperate to form a sealed environment. The heating member 64 can apply appropriate pressure to the reaction tubes 32. This will benefit heat conduction within the reaction tubes 32 and the sample block 31. In addition, the pressure exerted on the reaction tube 32 by the heating member 64 can also enhance the tightness of caps of the reaction tubes 32, so as to prevent the sample gas in the reaction tubes 32 from leaking and being contaminated. In addition, the heating member 64 can heat the air above the sample block 31 at a temperature higher than the reaction temperature, so as to prevent condensation of water vapor in the reaction tubes 32. This can ensure the concentrations of the reaction mixture in the reaction tubes 32 remain unchanged during the alternated heating and cooling cycle.

With continued reference to FIG. 2, in addition, a protocol running device port 35 is provided in the chamber 11 of the main body 10. The protocol running device port 35 electrically connects with a host system (not shown) of the main body 10, and is exposed on a surface of the chamber 11. The protocol running device port 35 can place a protocol running device 38 stored with a protocol. Through the electrical connection of the protocol running device port 35, the protocol stored in the external protocol running device 38 can be read by the protocol running device port 35 and then recognized by the host system of the main body 10. Based on the protocol, the host system sets environments and related parameters of the thermal cycle, and then performs a thermal cycle reaction to the samples in the reaction tubes 32 placed in the sample block 31. In an embodiment, the thermal cycler 2 is a polymerase chain reaction (PCR) instrument, and the protocol stored in the protocol running device 38 includes at least temperatures and durations for steps of the polymerase chain reaction. The host system of the main body includes a chip and related circuits with processing capabilities. Conventional PCR instruments include a user interface or keypads allowing the user to input or select temperatures and durations and sequence for the thermal cycle reaction. By contrast, the thermal cycler 2 provided by the present invention merely requires that the user puts the protocol running device 38 into the protocol running device port 35, and then after closing the lid 16 and pressing the start key 14, the thermal cycler 2 can start the polymerase chain reaction.

In addition, optionally, the protocol in the protocol running device 38 can also regulate the setting in response to an event of the lid 16 detected by the locking sensor 36. Examples may include but are not limited to, when the locking sensor 36 detects that the lid 16 is opened, the setting of the thermal cycle in execution is interrupted or continued.

With continued reference to FIG. 2, one advantage of the thermal cycler 2 of the present invention is that a user only needs to insert the protocol running device 38 stored with a protocol into the protocol running device port 35 and press the start key 14 of the main body 10, and then the thermal cycler 2 can perform and complete a thermal cycle reaction such as a polymerase chain reaction. In particular, the user may not have the manipulative skills or even the relevant knowledge of thermal cycling reactions. Protocols with setting corresponding to various samples can be pre-written in different protocol running devices 38. The protocol corresponding to the sample to be tested is firstly confirmed before running the reaction, to prevent the erroneous inputting/setting/selection that occurs in operation of the conventional thermal cyclers. In addition, because the thermal cycler 2 of the present invention is provided with a protocol running device port 35 that directly places the protocol running device 38, the thermal cycler 2 can be operated independently. The thermal cycler 2 is not restricted by environment of the use; the reactions can be run even in an environment without network or computer. Furthermore, the pre-written protocol running device 38 can reduce the overall time for performing the thermal cycle reaction and shorten the time for obtaining the reaction results and products.

In addition, the protocol running device 38 of the present invention may be implemented as an erasable programmable read only memory (EPROM), a flash memory, or a radio frequency identification (RFID) tag, etc., where a protocol may be pre-written into the device via matching. Depending on different conditions required by different polymerase chain reactions, the present invention can write the temperatures and durations required for the thermal cycle reaction and the related parameters into a programmable read-only memory or other appropriate storage devices in a form of protocol. The user is only required to put the protocol running device 38 corresponding to the polymerase chain reaction into the thermal cycler 2 of the present invention, and then the polymerase chain reaction can be started. In conventional instruments, a control interface of the instrument or a computer interface connected to the instrument is employed to input conditions for the thermal cycle reaction. Compared with conventional instruments, the present invention provides a division of labor that dividing works into two different stages: professionals design/input the conditions for the thermal cycle reaction, and operators of the instrument places the protocol running device 38 that match with the polymerase chain reaction into the thermal cycler 2 and then press the start key 14. This can ensure that correct reaction conditions are used.

In summary, the present invention provides a thermal cycler, which is simple and convenient to carry when it is applied in the field of polymerase chain reaction. As long as a protocol running device with a protocol is provided to an operator having ordinary skill in the art, the reaction can be carried out according to the present invention. With a pre-written protocol, general users can carry out a polymerase chain reaction in real time. This is beneficial to reduce the probability of an erroneous setting.

Accordingly, although an exemplary embodiment and method according to the invention have been shown and described, it is to be understood that all the terms used herein are descriptive rather than limiting, and that many changes, modifications, and substitutions may be made by one having ordinary skill in the art without departing from the spirit and scope of the invention.

1. A thermal cycler, comprising:
a main body comprising a chamber and a sample block in the chamber for placing a plurality of reaction tubes; and
a protocol running device port disposed in the chamber to place a protocol running device, wherein a user inserts the protocol running device into the protocol running device port, and the main body executes a thermal cycle on the reaction tubes according to a protocol stored in the protocol running device.

2. The thermal cycler as recited in claim 1, further comprising a lid that presses against the main body to seal the chamber between the lid and the main body.

3. The thermal cycler as recited in claim 2, wherein the lid comprises a heating member, and when the lid presses against the main body and reaction tubes are placed in the sample block, the heating member is located above the sample block and the reaction tubes to heat the sample block and to pressurize the reaction tubes.

4. The thermal cycler as recited in claim 3, wherein the lid comprises a locking sensor to detect whether the lid is closed or is locked with the main body.

5. The thermal cycler as recited in claim 4, wherein the protocol comprising a setting to interrupt or stop the thermal cycle in response to a detecting result of the locking sensor, or further comprising a setting of a plurality of temperatures and durations required for carrying out a polymerase chain reaction.

6. The thermal cycler as recited in claim 5, wherein the protocol running device comprises an erasable and programmable read-only memory, a flash memory, or a radio frequency identification tag to store the protocol.

7. The thermal cycler as recited in claim 1, wherein the protocol comprising a setting of a plurality of temperatures and durations required for carrying out a polymerase chain reaction.

8. The thermal cycler as recited in claim 7, wherein the protocol running device comprises an erasable and programmable read-only memory, a flash memory, or a radio frequency identification tag to store the protocol.

9. The thermal cycler as recited in claim 1, wherein the main body further comprises a main screen disposed on the main body to display the protocol, or the main body further comprises a main screen and a start key disposed on the main body, the main screen being used to display the protocol and the start key being used to start, interrupt, or stop the thermal cycle.

10. The thermal cycler as recited in claim 1, wherein the main body further comprises a start key disposed on the main body, and the start key is used to start, interrupt, or stop the thermal cycle.
